(19) United States
(12) Patent Application Publication
(10) Pub. No.: US 2025/0302387 A1
(43) Pub. Date: Oct. 2, 2025

(54) INSERTING APPARATUS FOR IMPLANTABLE SENSOR PLACEMENT

(71) Applicants: Jiangsu Yuwell POCTech Biotechnology Co., Ltd., Zhenjiang (CN); JIANGSU YUEKAI BIOTECHNOLOGY CO., LTD., Nanjing (CN); Zhejiang POCTech Co., Ltd., Huzhou (CN)

(72) Inventors: Jiajia ZHI, Nanjing (CN); Yanan ZHANG, Nanjing (CN)

(21) Appl. No.: 19/090,454

(22) Filed: Mar. 26, 2025

(30) Foreign Application Priority Data
Mar. 29, 2024 (CN) 202420648135.8
Jun. 28, 2024 (CN) 202410860523.7

(51) Int. Cl. A61B 5/00; A61B 5/145; A61B 90/00

(52) U.S. Cl. CPC A61B 5/6848; A61B 5/14503; A61B 5/14546; A61B 90/03; A61B 2090/034; A61B 2560/0462; A61B 2560/063

(57) ABSTRACT

An inserting apparatus for implantable sensor placement includes a housing, a monitoring unit, and an insertion unit. The monitoring unit is arranged in the housing and has a limiting portion. The insertion unit includes a driving assembly and a puncture assembly. The driving assembly is configured to drive the puncture assembly and the monitoring unit to move and includes a locking member configured to be engaged with the limiting portion. The locking member has a locked state in which the locking member abuts with the limiting portion and an unlocked state in which the locking member is separated from the limiting portion.

### CROSS-REFERENCE TO RELATED APPLICATION

This application is based on Chinese patent applications No. 202420648135.8 filed on Mar. 29, 2024 and No. 202410860523.7 filed on Jun. 28, 2024, and claims priorities to the above Chinese patent applications, the entire contents of which are incorporated herein by reference.

### FIELD

The present disclosure relates to the field of medical equipment technologies, and more particularly, to an inserting apparatus for implantable sensor placement.

### BACKGROUND

Detecting various analytes in an individual's body is crucial for monitoring health status of the individual. Deviations from normal analyte levels can often indicate potential physiological status, such as metabolic conditions, diseases, etc. Periodic ex vivo analyte monitoring using extracted body fluid is sufficient to observe the physiological status of many individuals. However, ex vivo analyte monitoring, due to the limited number of measurements, may miss critical measurement data, thereby causing a delay in optimal treatment timing and potentially causing irreversible harm to the patient. In addition, for individuals with severe analyte disorders and/or rapid fluctuations in analyte levels, frequent extraction of body fluid is required for monitoring, which will bring inconvenience and pain to the patients.

In many cases, subcutaneous, interstitial, or skin analyte sensors can provide sufficient measurement accuracy while minimizing discomfort of a user. Continuous analyte monitoring using an implantable analyte monitoring sensor is an ideal monitoring method. Typically, the analyte monitoring sensor is implanted into an individual's body by using an inserting apparatus. The sensor reacts with body fluid of an implanted person to generate an electrical signal, and a processing unit converts the electrical signal into data that can characterize analyte concentration. Then the data is transmitted to a display device for display. In this way, continuous monitoring of the analyte concentration is realized.

The detachable connection between the existing monitoring unit and the inserting apparatus is relatively unstable, which causes the monitoring unit to fall off during transportation or use of a product, reducing a success rate of implantation of the product.

### SUMMARY

To this end, an object of the present disclosure is to provide an inserting apparatus for implantable sensor placement. The inserting apparatus can improve a success rate of implantation.

According to a first aspect of the present disclosure, an inserting apparatus for implantable sensor placement is provided. The inserting apparatus includes a housing, a monitoring unit, and an insertion unit. The monitoring unit is arranged in the housing and has a limiting portion. The insertion unit includes a driving assembly and a puncture assembly. The driving assembly is configured to drive the puncture assembly and the monitoring unit to move and includes a locking member configured to be engaged with the limiting portion. The locking member has a locked state in which the locking member abuts with the limiting portion and an unlocked state in which the locking member is separated from the limiting portion.

With the inserting apparatus according to some embodiments of the present disclosure, the puncture assembly includes a fixing portion configured to apply a force towards the limiting portion to the locking member, to maintain the locking member in the locked state.

With the inserting apparatus according to some embodiments of the present disclosure, the housing has a first accommodation cavity with a downward opening and a second accommodation cavity located below the first accommodation cavity. The monitoring unit includes a sensor assembly disposed in the first accommodation cavity and a transmitter assembly disposed in the second accommodation cavity. The limiting portion is provided at the sensor assembly.

With the inserting apparatus according to some embodiments of the present disclosure, the puncture assembly and the sensor assembly have a first relative position in which the puncture assembly and the sensor assembly are engaged with each other and a second relative position in which the puncture assembly and the sensor assembly are separated from each other. In the first relative position, the fixing portion abuts against the locking member to restrict the locking member to move relative to the limiting portion. In the second relative position, the fixing portion is disengaged from the locking member to release a limitation on the locking member.

With the inserting apparatus according to some embodiments of the present disclosure, the transmitter assembly has a first engagement structure. The sensor assembly has a second engagement structure configured to be engaged with the first engagement structure, to connect the sensor assembly to the transmitter assembly.

With the inserting apparatus according to some embodiments of the present disclosure, the driving assembly includes a first drive member configured to drive the puncture assembly to move from an initial position to an insertion position, and a second drive member configured to drive the puncture assembly to move from the insertion position to a retraction position, to enable the fixing portion to be disengaged from the locking member.

With the inserting apparatus according to some embodiments of the present disclosure, a seal configured to seal the first accommodation cavity is disposed between the first accommodation cavity and the second accommodation cavity. The seal is removable through an aperture provided at a circumferential side of the housing, to enable the first accommodation cavity and the second accommodation cavity to be in communication with each other.

With the inserting apparatus according to some embodiments of the present disclosure, the locking member includes at least two locking portions. The fixing portion is configured to extend between the at least two locking portions, to prevent the at least two locking portions from moving relative to the limiting portion.

With the inserting apparatus according to some embodiments of the present disclosure, the locking member is an elastic member and is in a deformed state when the locking member is in the locked state.

With the inserting apparatus according to some embodiments of the present disclosure, the locking member includes a connection segment and a locking segment disposed at a circumferential side of the connection segment. The limiting portion includes a connection groove configured to accommodate the connection segment, and an engagement groove radially recessed from a groove wall of the connection groove and configured to accommodate the locking segment. In the locked state, the locking segment is located inside the engagement groove. In the unlocked state, the locking segment is disengaged from the engagement groove.

With the inserting apparatus according to some embodiments of the present disclosure, the limiting portion has a contact surface abutting against and engaged with the locking member. The contact surface is inclined from a center to a periphery in a movement direction of the monitoring unit.

According to a second aspect of the present disclosure, an inserting apparatus for implantable sensor placement is provided. According to some embodiments of the present disclosure, the inserting apparatus includes a housing, a monitoring unit, a driving assembly, and a seal. The housing has a first accommodation cavity and a second accommodation cavity located below the first accommodation cavity. The housing has an aperture provided at a circumferential side of the housing and in communication with the first accommodation cavity and/or the second accommodation cavity. The monitoring unit includes a sensor assembly disposed in the first accommodation cavity and a transmitter assembly disposed in the second accommodation cavity. The driving assembly is configured to drive the monitoring unit to move and includes a locking member configured to prevent the monitoring unit from being disengaged from the driving assembly. The seal is configured to seal the first accommodation cavity. The seal is removable through the aperture to unseal the first accommodation cavity.

With the inserting apparatus according to some embodiments of the present disclosure, steps of assembling the transmitter assembly and the sensor assembly can be omitted to simplify operation steps required when the inserting apparatus is used, in such a manner that use experience of a user is improved and an operation difficulty of the user is reduced. In this way, the user can quickly mount the inserting apparatus to the skin, which shortens an operation time of the user and reduces a risk of the sensor assembly being exposed to air and being contaminated, avoiding a risk of the user being infected.

With the inserting apparatus according to some embodiments of the present disclosure, the seal includes a manipulating portion extending to an outside of the housing via the aperture.

With the inserting apparatus according to some embodiments of the present disclosure, the seal is a diaphragm structure bonded to an opening of the first accommodation cavity. The manipulating portion extends from the aperture to the outside of the housing.

With the inserting apparatus according to some embodiments of the present disclosure, the housing includes an upper housing and a base connected to the upper housing.

The first accommodation cavity is formed inside the upper housing. The second accommodation cavity is formed inside the base. The upper housing has a sealing surface at a bottom of the upper housing. The seal is connected to the sealing surface to seal the first accommodation cavity.

With the inserting apparatus according to some embodiments of the present disclosure, the base has a connection area and an avoidance area in a circumferential direction of the base. The connection area is provided with a connection rib connected to the upper housing. The aperture is formed between the avoidance area and the upper housing.

With the inserting apparatus according to some embodiments of the present disclosure, a height H of the aperture and a thickness D of the seal satisfy: H≥2D.

According to a third aspect of the present disclosure, an inserting apparatus for implantable sensor placement is provided. According to some embodiments of the present disclosure, the inserting apparatus includes a housing, a monitoring unit, a puncture assembly, and a driving assembly. The housing includes an upper housing and a lower housing detachably connected to the upper housing. The monitoring unit has an outer circumferential surface sealingly connected to an inner circumferential surface of the lower housing. The sealed cavity is formed by the lower housing and the monitoring unit. The puncture assembly includes a seat and a needle disposed at the seat. The needle has an insertion portion arranged around the sensor assembly. Both the insertion portion and the sensor assembly are located in the sealed cavity. The driving assembly is configured to drive the puncture assembly and the monitoring unit to move.

With the inserting apparatus according to some embodiments of the present disclosure, the lower housing is provided with a sealing rib located at the inner circumferential surface of the lower housing and extending in a circumferential direction of the monitoring unit. The sealing rib has an inner circumferential surface sealingly connected to the outer circumferential surface of the monitoring unit.

With the inserting apparatus according to some embodiments of the present disclosure, the transmitter assembly has a limiting portion. The upper housing has a core cylinder and a sleeve. The driving assembly is configured to drive the sleeve and the monitoring unit to move through the core cylinder. The core cylinder has a locking member configured to extend into the limiting portion. The sleeve has a fixing portion acting on the locking member, to enable the locking member to have a locked state in which the locking member abuts against the limiting portion and an unlocked state in which the locking member is separated from the limiting portion.

With the inserting apparatus according to some embodiments of the present disclosure, before insertion, the locking member remains in the locked state, and abuts against the limiting portion to lock the monitoring unit to the driving assembly, in such a manner that connection stability between the monitoring unit and the insertion unit is enhanced, improving the success rate of implantation. After the insertion, the locking member is switched to the unlocked state to disengage the locking member from the limiting portion, in such a manner that the limitation on the locking member is released, releasing the monitoring unit from the driving assembly. In this way, smooth separation between the monitoring unit and the driving assembly is ensured when a needle is withdrawn.

Additional aspects and advantages of the present disclosure will be provided in part in the following description, or will become apparent in part from the following description, or can be learned from practicing of the present disclosure.

### BRIEF DESCRIPTION OF THE DRAWINGS

FIG. 1 is a schematic structural view of an inserting apparatus for implantable sensor placement according to an embodiment of the present disclosure.

FIG. 2 is a cross-sectional view of an inserting apparatus for implantable sensor placement according to an embodiment of the present disclosure.

FIG. 3 is an enlarged view of portion A in FIG. 2.

FIG. 4 is a schematic structural view of a core cylinder according to an embodiment of the present disclosure.

FIG. 5 is a schematic structural view of a sensor assembly according to an embodiment of the present disclosure.

FIG. 6 is a schematic structural view of an inserting apparatus for sensor implantable sensor according to an embodiment of the present disclosure, viewed from another perspective.

FIG. 7 is an enlarged view of portion B in FIG. 6.

FIG. 8 is a partial schematic structural view of an inserting apparatus for implantable sensor placement according to an embodiment of the present disclosure.

FIG. 9 is a partial schematic structural view of an inserting apparatus for implantable sensor placement according to an embodiment of the present disclosure.

FIG. 10 is a schematic structural view of an inserting apparatus for implantable sensor placement according to another embodiment of the present disclosure.

FIG. 11 is a cross-sectional view of an inserting apparatus for implantable sensor placement according to another embodiment of the present disclosure.

FIG. 12 is an enlarged view of portion C in FIG. 11.

FIG. 13 is a schematic exploded structural view of an inserting apparatus for implantable sensor placement according to another embodiment of the present disclosure.

FIG. 14 is a schematic structural view of a bottom cover according to another embodiment of the present disclosure.

FIG. 15 is a cross-sectional view of an inserting apparatus for implantable sensor placement according to another embodiment of the present disclosure, viewed from another perspective.

FIG. 16 is an enlarged view of portion D in FIG. 15.

FIG. 17 is a schematic structural view of a second seal according to another embodiment of the present disclosure.

FIG. 18 is a schematic structural view of a monitoring unit according to another embodiment of the present disclosure.

FIG. 19 is a schematic structural view of a cover body according to another embodiment of the present disclosure.

FIG. 20 is a schematic structural view of a core according to another embodiment of the present disclosure.

Reference numerals of the accompanying drawings:
1, housing; 11, upper housing; 111, first accommodation cavity; 112, seal; 113, body; 114, manipulating portion;
115, aperture, 116, edge; 117, flange;
12, base; 121, second accommodation cavity; 122, mounting seat; 123, limit engagement portion;
13, lower housing; 131, sealing rib; 132, first seal, 133, body portion; 134, sealing portion; 135, sleeve; 136, stop cylinder; 137, support column; 138, sealed cavity;
2, monitoring unit; 21, sensor assembly; 211, engagement portion; 212, second engagement structure;
22, transmitter assembly; 221, first engagement structure; 222, cover body; 223, fixing sticker; 224, limiting hole; 225, mounting groove; 226, accommodation groove;
23, limiting portion; 231, connection groove; 232, engagement groove; 31, trigger button; 32, trigger ring;
4, driving assembly; 41, first drive member; 42, second drive member; 43, core cylinder; 431, locking member; 432, connection segment; 433, locking segment; 434, positioning portion;
5, puncture assembly; 51, seat; 511, second seal; 512, limit connection portion; 513, connection segment; 514, stop segment; 52, needle; 53, sleeve; 54, fixing portion; 6, protection member.

### DETAILED DESCRIPTION

Embodiments of the present disclosure will be described in detail below with reference to examples thereof as illustrated in the accompanying drawings, throughout which same or similar elements, or elements having same or similar functions, are denoted by same or similar reference numerals. The embodiments described below with reference to the drawings are illustrative only, and are intended to explain, rather than limit, the present disclosure.

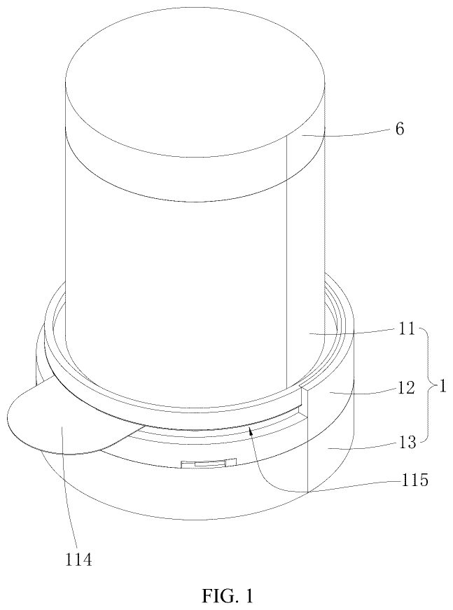

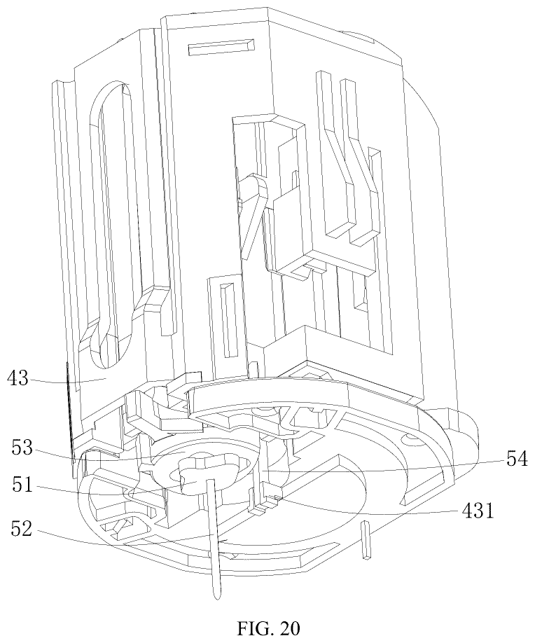

As illustrated in FIG. 1 to FIG. 20, an inserting apparatus for implantable sensor placement is provided. The inserting apparatus includes a housing 1, a monitoring unit 2, and an insertion unit. The monitoring unit 2 is arranged in the housing 1 and has a limiting portion 23. The insertion unit includes a driving assembly 4 and a puncture assembly 5. The driving assembly 4 is configured to drive the puncture assembly 5 and the monitoring unit 2 to move and includes a locking member 431 configured to be engaged with the limiting portion 23. The locking member 431 has a locked state in which the locking member 431 abuts with the limiting portion 23 and an unlocked state in which the locking member 431 is separated from the limiting portion 23.

When the inserting apparatus is not mounted to a human body, the locking member 431 remains in the locked state, and abuts with the limiting portion 23 to lock the monitoring unit 2 to the driving assembly 4, in such a manner that connection stability between the monitoring unit 2 and the insertion unit is enhanced, and probability of the monitoring unit 2 shifting or falling off during transportation and use is reduced, improving a success rate of implantation. After the inserting apparatus is mounted to the human body, the driving assembly 4 is configured to drive the puncture assembly 5 and the monitoring unit 2 to move, in such a manner that the monitoring unit 2 is attached to the human body. The locking member 431 is switched to the unlocked state to disengage the locking member 431 from the limiting portion 23, in such a manner that a limitation on the locking member 431 is released, allowing the monitoring unit 2 to be released from the driving assembly 4. In this way, smooth separation between the monitoring unit 2 and the driving assembly 4 during a needle withdrawn is ensured, improving use experience of a user.

As illustrated in FIG. 1 to FIG. 9, the inserting apparatus includes the housing 1, the monitoring unit 2, and the insertion unit. The housing 1 has a first accommodation cavity 111 with a downward opening and a second accommodation cavity 121 located below the first accommodation cavity 111. The monitoring unit 2 includes a sensor assembly 21 arranged in the first accommodation cavity 111 and a transmitter assembly 22 arranged in the second accommodation cavity 121. The limiting portion 23 is provided at the sensor assembly 21. The insertion unit includes the driving assembly 4 and the puncture assembly 5 that are arranged in the first accommodation cavity 111. The driving assembly 4 is configured to drive the puncture assembly 5 to move, to drive the sensor assembly 21 to be mounted at the transmitter assembly 22. The driving assembly 4 includes the locking member 431 configured to extend into the limiting portion 23. The puncture assembly 5 includes a fixing portion 54 configured to press the locking member 431. The puncture assembly 5 and the sensor assembly 21 have a first relative position in which the puncture assembly 5 is adjacent to the sensor assembly 21 and a second relative position in which the puncture assembly 5 and the sensor assembly 21 are separated from each other. In the first relative position, the fixing portion 54 abuts against the locking member 431 to restrict the locking member 431 to move relative to the limiting portion 23, in such a manner that the sensor assembly 21 is locked to the driving assembly 4. In the second relative position, the fixing portion 54 is disengaged from the locking member 431 to release the limitation on the locking member 431, in such a manner that the sensor assembly 21 is released from the driving assembly 4.

It should be noted that, the first relative position in the present disclosure refers to a position of the puncture assembly 5 and the sensor assembly 21 when the fixing portion 54 comes into contact with and presses against the locking member 431 and the fixing portion 54 extends into the limiting portion 23. At this time, the locking member 431 is in the locked state. The second relative position refers to a position of the puncture assembly 5 and the sensor assembly 21 when the fixing portion 54 is separated from the locking member 431 and disengaged from the limiting portion 23. At this time, the locking member 431 is in the unlocked state.

When the inserting apparatus in the present disclosure is not mounted to the human body, the puncture assembly 5 and the sensor assembly 21 are in the first relative position, in such a manner that the fixing portion 54 abuts against the locking member 431, and the locking member 431 is limited in the limiting portion 23, and the locking member 431 is in the locked state. In this way, the sensor assembly 21 is locked to the driving assembly 4, enhancing the connection stability between the online monitoring unit 2 and the insertion unit. After the inserting apparatus is mounted to the human body, the puncture assembly 5 and the sensor assembly 21 are in the second relative position, in such a manner that the fixing portion 54 is disengaged from the locking member 431 to release the limitation on the locking member 431, and the locking member 431 is in the unlocked state. In this way, the sensor assembly 21 is released from the driving assembly 4, ensuring that the sensor assembly 21 and the driving assembly 4 are smoothly separated during the needle withdrawn.

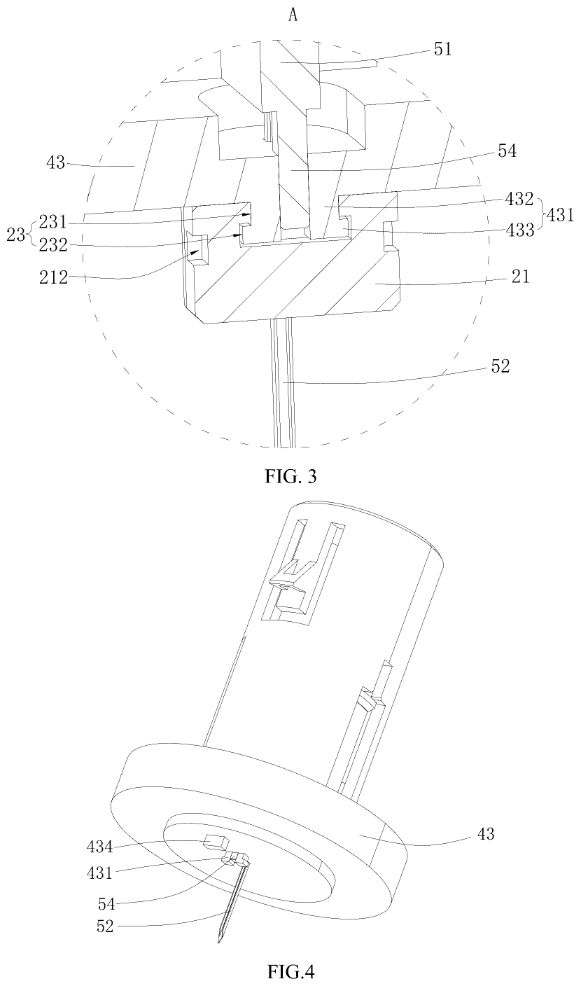

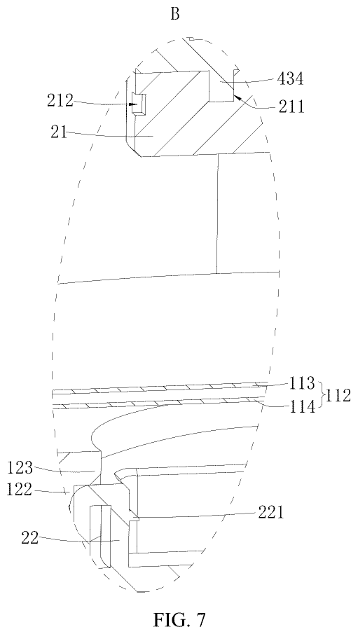

The present disclosure does not specifically limit a structure of the fixing portion 54. Preferably, as illustrated in FIG. 3, FIG. 4, and FIG. 7, the fixing portion 54 is a columnar structure with a polygonal cross-section, which increases contact area between the fixing portion 54 and the locking member 431, in such a manner that a pressing effect of the fixing portion 54 on the locking member 431 is enhanced. In other embodiments, the fixing portion 54 may further be a columnar structure with a circular or other shaped cross section.

The present disclosure does not specifically limit a relative position between the limiting portion 23 and the sensor assembly 21, and any one of the following embodiments may be adopted.

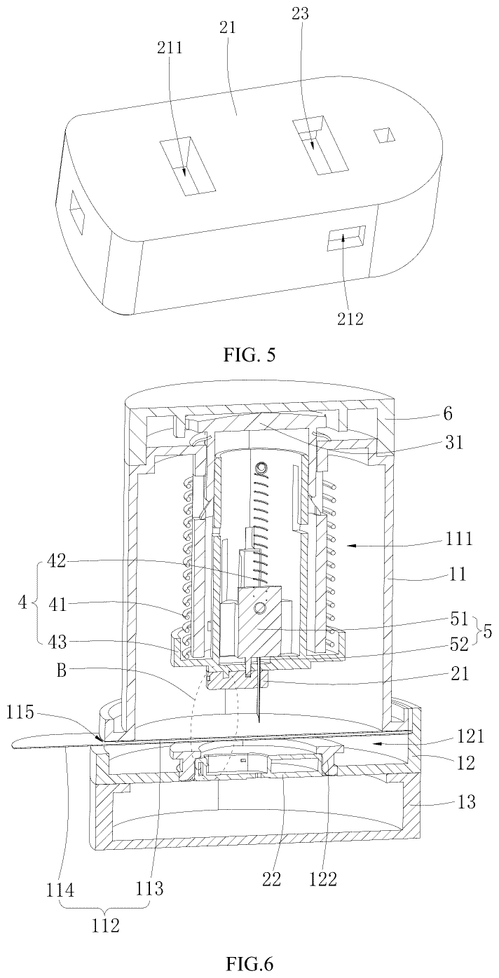

Embodiment 1: in this embodiment, as illustrated in FIG. 5, a top surface of the sensor assembly 21 has a central area and an edge area surrounding the central area. The limiting portion 23 is arranged at the central area.

Since the limiting portion 23 is provided at the central area of the sensor assembly 21, the locking member 431 and the fixing portion 54 can avoid the transmitter assembly 22, which enhances smoothness of assembling the sensor assembly 21 and the transmitter assembly 22 together. In addition, the number of the locking member 431 and the fixing portion 54 can be reduced to simplify a structure of the inserting apparatus, in such a manner that a production cost of the inserting apparatus is reduced. In addition, the limiting portion 23 is provided at the central area of the sensor assembly 21, which can balance a force acting on the sensor assembly 21 to enhance stability of the sensor assembly 21. In this way, the smoothness of assembling the sensor assembly 21 and the transmitter assembly 22 together is further enhanced.

In this embodiment, a structure of the locking member 431 is not specifically limited. Preferably, the locking member 431 includes at least two locking portions. The fixing portion 54 can extend between the at least two locking portions, to limit the at least two locking portions in the limiting portion 23.

Since the at least two locking portions are provided, connection points between the locking portion 431 and the sensor assembly 21 are increased to enhance connection stability between the sensor assembly 21 and the driving assembly 4. The fixing portion 54 can extend between the at least two locking portions to enable the single fixing portion 54 to press a plurality of locking portions, to simplify the structure of the fixing portion 54 and achieve an effect that the sensor assembly 21 is easily assembled to the driving assembly 4, improving production efficiency of the inserting apparatus.

The present disclosure does not specifically limit the number of locking portions. Preferably, as illustrated in FIG. 3 and FIG. 4, two locking portions are provided and are respectively located at two opposite sides of the fixing portions 54, which enhances the pressing effect of the fixing portion 54 on the locking portions, ensuring the connection stability between the sensor assembly 21 and the driving assembly 4. In this way, a risk of the sensor assembly 21 being separated from the driving assembly 4 during the transportation or handling is reduced, and product quality of the inserting apparatus is further improved. In other embodiments, three, four, or different numbers of the locking portion may be provided along a circumferential side of the fixing portion 54.

Embodiment 2: in this embodiment, the top surface of the sensor assembly 21 has the central area and the edge area surrounding the central area. The limiting portion 23 is provided at the edge area. A plurality of limiting portions 23 are arranged along a circumferential side of the sensor assembly 21. A plurality of fixing portions 54 and a plurality of locking members 431 are arranged corresponding to the plurality of limiting portions 23. Each fixing portion 54 is located at a side of the corresponding locking member 431 away from the sensor assembly 21. In addition, the transmitter assembly 22 has an avoidance groove corresponding to the fixing portion 54 and the locking members 431, in such a manner that when the sensor assembly 21 and the transmitter assembly 22 are assembled together, the avoidance groove avoids the fixing portion 54 and the locking member 431 to ensure the smoothness of assembling the sensor assembly 21 and the transmitter assembly 22 together.

In a preferred embodiment, the locking member 431 is an elastic member and is in a deformed state when the locking member 431 is in the locked state.

That is, the locking member 431 is made of elastic material, for example, elastic plastic or elastic metal material.

Since the locking member 431 is the elastic member, an elastic swing effect of the locking member 431 is enhanced, and an ability of the elastic member to recover from deformation after the fixing portion 54 is separated from the elastic member is increased, which further improves smoothness of the sensor assembly 21 being separated from the driving assembly 4 during the needle withdrawn.

The present disclosure does not specifically limit a limit engagement method between the locking member 431 and the limiting portion 23, and any one of the following embodiments may be adopted.

Embodiment 1: in this embodiment, as illustrated in FIG. 3 and FIG. 4, the locking member 431 includes a connection segment 432 and a locking segment 433 disposed at a circumferential side of the connection segment 432. The limiting portion 23 includes a connection groove 231 configured to accommodate the connection segment 432, and an engagement groove 232 arranged at a groove wall of the connection groove 231 and configured to accommodate the locking segment 433. In the first relative position, the locking segment 433 is located inside the engagement groove 232. In the second relative position, the locking segment 433 is disengaged from the engagement groove 232.

In other words, in the first relative position, the locking member 431 is engaged with the limiting portion 23 to limit the locking member 431 in the limiting portion 23, in such a manner that connection stability between the locking member 431 and the limiting portion 23 is improved to enhance a fixation effect of the sensor assembly 21, and to reduce possibility of the sensor assembly 21 being separated from the driving assembly 4 due to vibration during the transportation or the handling of the inserting apparatus. In this way, quality stability of the inserting apparatus is improved, and the use experience of the user is further improved.

Embodiment 2: in this embodiment, the limiting portion 23 has a contact surface abutting against and engaged with the locking member 431. The contact surface is inclined from top to bottom and from a center to a periphery.

In other words, the locking member 431 is engaged with and abuts against the groove wall of the limiting portion 23. Since the contact surface is inclined from top to bottom and from the center to the periphery, notch area of the limiting portion 23 is smaller than bottom area of the limiting portion 23, in such manner that when the fixing portion 54 is located in the limiting portion 23, the sensor assembly 21 cannot be separated from the driving assembly 4, enhancing the fixation effect of the sensor assembly 21 and reducing a difficulty of molding the limiting portion 23 and the locking member 431. In this way, the production cost of the inserting apparatus is reduced and the production efficiency thereof is improved.

In an exemplary embodiment of the present disclosure, when the fixing portion 54 extends into the limiting portion 23, the fixing portion 54 presses the locking member 431 to elastically deform the locking member 431 in a direction away from the fixing portion 54, in such a manner that the locking member 431 abuts against the contact surface. However, when the fixing portion 54 is disengaged from the limiting portion 23, the locking member 431 gradually recovers from the deformation, in such a manner that an end surface of the locking member 431 opposite to the contact surface is in a vertical state or inclined from top to bottom in a direction away from the contact surface. In this way, the locking member 431 can be separated from the sensor assembly 21 after the fixing portion 54 is disengaged from the limiting portion 23.

It should be noted that, the contact surface is inclined from top to bottom and from the center to the periphery means that the contact surface is inclined from top to bottom in a direction away from the locking member 431.

In a preferred embodiment, as illustrated in FIG. 4, FIG. 5 and FIG. 7, a bottom of the driving assembly 4 is provided with a positioning portion 434. A top of the sensor assembly 21 is provided with an engagement portion 211. The positioning portion 434 is configured to be engaged with the engagement portion 211 to position the sensor assembly 21.

After the sensor assembly 21 and the driving assembly 4 are assembled together, the positioning portion 434 is engaged with the engagement portion 211 to position the sensor assembly 21, which enhances the stability of the sensor assembly 21 and avoids a situation that the sensor assembly 21 and the transmitter assembly 22 may be misaligned. In this way, the smoothness of assembling the sensor assembly 21 and the transmitter assembly 22 together is ensured to reduce an operation difficulty of the user, and the use experience of the user is further improved.

The present disclosure does not specifically limit structures of the positioning portion 434 and the engagement portion 211. Preferably, as illustrated in FIG. 4, FIG. 5, and FIG. 7, the positioning portion 434 is a block structure disposed at the bottom of the driving assembly 4. The engagement portion 211 is a groove structure disposed at the top surface of the sensor assembly 21. In this way, the block structure is inserted into and engaged with the groove structure to position the sensor assembly 21. In other embodiments, the positioning portion 434 may further be a groove structure disposed at the bottom of the driving assembly 4. The engagement portion 211 may further be a block structure disposed at the top surface of the sensor assembly 21. Alternatively, the positioning portion 434 is an annular rib disposed at the bottom of the driving assembly 4 and extending in a circumferential direction of the sensor assembly 21, and the engagement portion 211 is an annular notch disposed at the top surface of the sensor assembly 21 to accommodate the annular rib.

The present disclosure does not specifically limit a connection method between the sensor assembly 21 and the transmitter assembly 22, and any one of the following embodiments may be adopted.

Embodiment 1: in this embodiment, as illustrated in FIG. 3, FIG. 5, and FIG. 7, the transmitter assembly 22 has a first engagement structure 221. The sensor assembly 21 has a second engagement structure 212 configured to be engaged with the first engagement structure 221, to connect the sensor assembly 21 to the transmitter assembly 22.

After the sensor assembly 21 and the transmitter assembly 22 are assembled together, the first engagement structure 221 is engaged with the second engagement structure 212 to enhance connection stability between the sensor assembly 21 and the transmitter assembly 22, which avoids a situation that the sensor assembly 21 and the transmitter assembly 22 can move relative to each other to cause the user to feel discomfort. In addition, the product quality of the inserting apparatus can be improved, and the use experience of the user is further improved.

The present disclosure does not specifically limit structures of the first engagement structure 221 and the second engagement structure 212. Preferably, the first engagement structure 221 is a protrusion disposed inside of the transmitter assembly 22. The second engagement structure 212 is a groove formed at the circumferential side of the sensor assembly 21. The protrusion is engaged with the groove to connect the sensor assembly 21 to the transmitter assembly 22, in such a manner that the connection stability between the sensor assembly 21 and the transmitter assembly 22 is further enhanced.

Preferably, the protrusion is elastic, allowing the protrusion to swing relative to the transmitter assembly 22, which ensures the smoothness of assembling the sensor assembly 21 and the transmitter assembly 22 together.

In other embodiments, the first engagement structure 221 may further be a groove formed inside the transmitter assembly 22. The second engagement structure 212 may be a protrusion disposed at the circumferential side of the sensor assembly 21.

The present disclosure does not specifically limit the number of the first engagement structure 221 and the second engagement structure 212. Preferably, a plurality of first engagement structures 221 are provided at intervals in a circumferential direction of an inner wall of the transmitter assembly 22, and a plurality of second engagement structures 212 are provided corresponding to the first engagement structures 221 to increase the number of fixing points for the sensor assembly 21, and thus the connection stability between the sensor assembly 21 and the transmitter assembly 22 is further enhanced. In other embodiments, one first engagement structure 221 and one second engagement structure 212 may correspondingly be provided.

Embodiment 2: in this embodiment, after the sensor assembly 21 and the transmitter assembly 22 are assembled together, a peripheral surface of the sensor assembly 21 abuts against the inner wall of the transmitter assembly 22, that is, the sensor assembly 21 is connected to the transmitter assembly 22 in an interference fit manner.

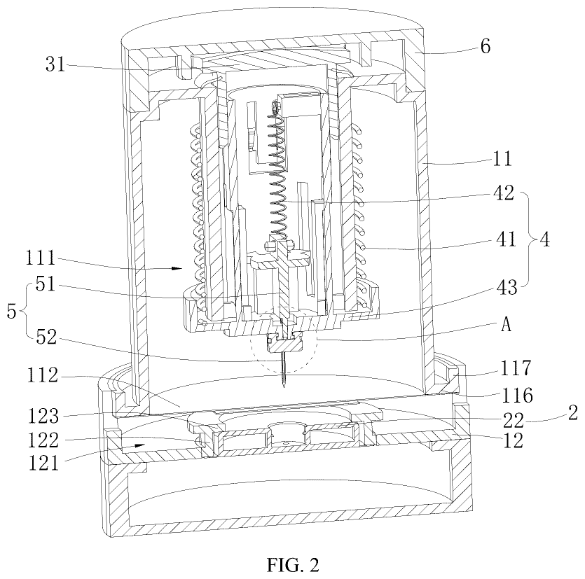

In a preferred embodiment, as illustrated in FIG. 2 and FIG. 6, the driving assembly 4 includes a first drive member 41 and a second drive member 42. The first drive member 41 is configured to drive the puncture assembly 5 to move from an initial position to an insertion position, to drive the sensor assembly 21 to be mounted at the transmitter assembly 22. The second drive member 42 is configured to drive the puncture assembly 5 to move from the insertion position to a retraction position, to enable the fixing portion 54 to be disengaged from the locking member 431.

When the inserting apparatus is mounted to the human body, the puncture assembly 5 is driven by the first drive member 41 to move from the initial position to the insertion position, to drive the sensor assembly 21 to be mounted at the transmitter assembly 22, and enable a part of sensors of the sensor assembly 21 to be pierced into the subcutaneous tissue. At the same time, a limitation on the puncture assembly 5 is released by the driving assembly 4. Then the puncture assembly 5 is driven by the second drive member 42, and the fixing portion 54 is separated from the locking member 431 to release the limitation on the locking member 431. The locking member 431 can be separated from the limiting portion 23, to enable the puncture assembly 5 to move from the insertion position to the retraction position. In this way, the sensor assembly 21 and the transmitter assembly 22 are mounted to the human body, and the needle 52 is withdrawn. In this process, the user only needs to trigger the driving assembly 4, in such a manner that the inserting apparatus in the present disclosure is simple to operate, which reduces the operation difficulty of the user to improve the use experience of the user.

In a preferred embodiment, as illustrated in FIG. 2 and FIG. 6, the driving assembly 4 further includes a core cylinder 43. The puncture assembly 5 is connected to the core cylinder 43. The locking member 431 and the positioning portion 434 are disposed at a bottom of the core cylinder 43. The first drive member 41 acts on the core cylinder 43 to allow the core cylinder 43 to drive the puncture assembly 5 to move from the initial position to the insertion position, which further enables the sensor assembly 21 to be assembled to the transmitter assembly 22.

In a preferred embodiment, as illustrated in FIG. 2 and FIG. 6, the puncture assembly 5 includes a seat 51 disposed inside the core cylinder 43 and a needle 52 disposed at the seat 51 and configured to extend out of the core cylinder 43. The fixing portion 54 is disposed at a bottom of the seat 51. The second drive member 42 is disposed inside the core cylinder 43 and is configured to drive the seat 51. In this way, the fixing portion 54 and the needle 52 are driven by the seat 51 to move from the insertion position to the retraction position.

The present disclosure does not specifically limit a structure of the first drive member 41. Preferably, as illustrated in FIG. 2 and FIG. 6, the first drive member 41 is a spring arranged around the core cylinder 43 to enhance a driving effect on the core cylinder 43. In other embodiments, the first drive member 41 may further be an elastic piece having an end acting on the core cylinder 43 and another end acting on the housing 1.

The present disclosure does not specifically limit a structure of the second drive member 42. Preferably, as illustrated in FIG. 2 and FIG. 6, the second drive member 42 is a tension spring having an end acting on the seat 51 and another end acting on the core cylinder 43, to enhance a driving effect on the seat 51. In other embodiments, the second drive member 42 may further be a rubber band.

In a preferred embodiment, as illustrated in FIG. 1, FIG. 2 and FIG. 6, the inserting apparatus further includes a trigger button 31 configured to trigger the driving assembly 4, and a protection member 6 detachably connected to the housing 1 and acting on the trigger button 31. The trigger button 31 can trigger the driving assembly 4 after the protection member 6 is removed.

Before the inserting apparatus is mounted to the human body, the protection member 6 is removed from the housing 1, and then the trigger button 31 is pressurized, in such a manner that the driving assembly 4 is triggered by the trigger button 31 to enable the puncture assembly 5 to be driven by the driving assembly 4. In this way, the sensor assembly 21 and the transmitter assembly 22 are assembled together and a sensor of the sensor assembly 21 pierces into the subcutaneous tissue. In addition, a protection member can avoid the trigger button 31 from being mistriggered, to improve the product quality of the inserting apparatus and reduce a damage rate of the inserting apparatus during the transportation, the handling and the use.

The present disclosure does not specifically limit a structure of the protection member 6. Preferably, as illustrated in FIG. 1, FIG. 2, and FIG. 6, the protection member 6 is a protection cap detachably connected to the housing 1 and covering the trigger button 31 to avoid false triggering. In other embodiments, the protection member 6 may further be an annular structure which is arranged around the trigger button, detachably connected to the housing 1, and has an opening.

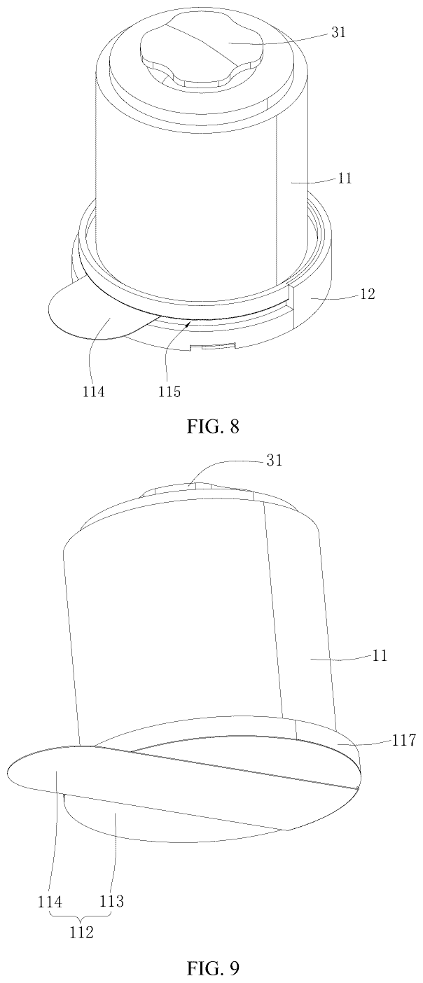

In a preferred embodiment, as illustrated in FIG. 1, FIG. 2, FIG. 6, FIG. 8, and FIG. 9, a seal 112 configured to seal the first accommodation cavity 111 is arranged at an opening of the first accommodation cavity. The housing 1 has an aperture 115 at a circumferential side of the housing 1 and located between the first accommodation cavity 111 and the second accommodation cavity 121. The seal 112 is removable from the opening of the first accommodation cavity 111 through the aperture 115, in such a manner that the first accommodation cavity 111 and the second accommodation cavity 121 are in communication with each other, which enables the sensor assembly 21 to be assembled to the transmitter assembly 22.

Since the first accommodation cavity 111 is sealed by the seal 112 and the sensor assembly 21 is disposed in the first accommodation cavity 111, a risk of contamination of the sensor assembly 21 with pathogenic bacteria is reduced to lower an infection risk of the user. In addition, the seal 112 is removable from the opening of the first accommodation cavity 111 through the aperture 115, which achieves an effect of facilitating the user to remove the seal 112. In this way, the operation difficulty of the user is reduced, and the use experience of the user is further improved.

When using the inserting apparatus in the present disclosure, the seal 112 is removed through the aperture 115 to unseal the first accommodation cavity 111. Then the insertion unit is triggered to move the sensor assembly 21 in a direction close to the transmitter assembly 22, in such a manner that the sensor assembly 21 is at least partially pierced into the subcutaneous tissue of the human body, the sensor assembly 21 and the transmitter assembly 22 are assembled together, and thus steps of assembling the transmitter assembly 22 and the sensor assembly 21 are omitted, operation steps required when using the inserting apparatus is simplified, improving the use experience of the user.

In a preferred embodiment, as illustrated in FIG. 1, FIG. 2, FIG. 6, FIG. 8, and FIG. 9, the seal 112 includes an operating portion 114 extending to an outside of the housing 1 via the aperture 115, to enable the seal 112 to be removed via the aperture 115 under a pulling action of the operating portion 114.

In other words, the seal 112 includes a body 113 and the manipulating portion 114 extending to the outside of the housing 1 via the aperture 115, in such a manner that when the first accommodation cavity 111 is unsealed, the manipulating portion 114 is pulled to drive the body 113 to move, and then the body 113 is separated from the opening of the first accommodation cavity 111. In addition, the body 113 is removed through the aperture 115 to complete unsealing of the first accommodation cavity 111, and the manipulating portion 114 only needs to be pulled during the unsealing. In this way, operation complexity of the user to unseal the first accommodation cavity 111 is simplified, in such a manner that the user can quickly unseal the first accommodation cavity 111, improving the use experience of the user.

The present disclosure does not specifically limit a structure of the seal 112, and any one of the following examples may be adopted.

First example: in this example, as illustrated in FIG. 1, FIG. 2, FIG. 6, FIG. 8, and FIG. 9, the seal 112 is a diaphragm structure bonded to the opening of the first accommodation cavity 111. The manipulating portion 114 extends from a side of the seal 112 away from the aperture 115 to the outside of the housing 1 through the aperture 115.

Since the seal 112 is bonded to the opening of the first accommodation cavity 111, a sealing effect of the seal 112 on the first accommodation cavity 111 is increased to ensure a sealing effect on the sensor assembly 21, avoiding contamination of the sensor assembly 21. In addition, the manipulating portion 114 extends from the side of the seal 112 away from the aperture 115 to the outside of the housing 1 through the aperture 115, in such a manner that when the manipulating portion 114 is pulled, the side of the seal 112 away from the aperture 115 is separated from the opening of the first accommodation cavity 111 firstly to reduce a difficulty of removing the seal 112 and further improve the use experience of the user.

In this embodiment, material of the seal 112 is not specifically limited. Preferably, the seal 112 is a diaphragm structure made of aluminum material to improve convenience of removing the seal 112. In other embodiments, the seal 112 may further be a diaphragm structure made of plastic or other metallic materials.

In the present disclosure, a bonding method between the seal 112 and the opening of the first accommodation cavity 111 is not specifically limited. Preferably, the seal 112 is connected to the opening of the first accommodation cavity 111 by a heat sealing process, to reduce a difficulty of assembling the seal 112 and improve the production efficiency of the inserting apparatus. In other embodiments, the seal 112 may further be bonded to the opening of the first accommodation cavity 111 by glue.

Second example: in this example, the seal 112 is a plate structure abutting with the opening of the first accommodation cavity 111. The plate structure has a sealing ring abutting against the opening of the first accommodation cavity 111, to enhance the sealing effect of the seal 112 on the first accommodation cavity 111 and achieve the effect of facilitating the user to remove the seal 112.

Third example: in this example, the seal 112 is a plate structure coming into contact with the opening of the first accommodation cavity 111. The plate structure is provided with a sealing portion extending into the first accommodation cavity 111 and abutting against an inner wall of the first accommodation cavity 111, to seal the first accommodation cavity 111.

The present disclosure does not specifically limit a structure of the housing 1. Preferably, as illustrated in FIG. 1, FIG. 2, and FIG. 6, the housing 1 includes an upper housing 11 and a base 12 connected to the upper housing 11. The first accommodation cavity 111 is formed inside the upper housing 11. The second accommodation cavity 121 is formed inside the base 12. The upper housing 11 has a sealing surface for the accommodation cavity 111 at a bottom of the upper housing 11. The seal 112 is connected to the sealing surface to seal the first accommodation cavity 111.

In other words, the housing 1 is a split structure, and since the first accommodation cavity 111 is formed at the upper housing 11 and the second accommodation cavity 121 is formed at the base 12, an effect of separately sterilizing the sensor assembly 21 and the transmitter assembly 22 can be achieved. In addition, sealing of the sterilized sensor assembly 21 can be facilitated.

Since the seal 112 is connected to the sealing surface, connection area between the seal 112 and the upper housing 11 is increased, which enhances stability of the seal 112. In this way, the sealing effect of the seal 112 on the first accommodation cavity 111 is ensured, and a difficulty of mounting the seal 112 is reduced.

The present disclosure does not specifically limit a formation method of the aperture 115. Preferably, the base 12 has a connection area and an avoidance area along a circumferential direction of the base 12, the connection area is provided with a connection rib connected to the upper housing 11, and the aperture 115 is formed between the avoidance area and the upper housing 11, in such a manner that production steps of the inserting apparatus are simplified, improving the production efficiency of the inserting apparatus. In addition, the amount of materials used for the inserting apparatus is reduced to reduce the production cost of the inserting apparatus.

Preferably, each of the connection area and the avoidance area occupies ½ of the base 12 in a circumferential direction of the base 12, to ensure smooth removal of the seal 112 and reduce the difficulty of removing the seal 112. In this way, the use experience of the user is improved.

The present disclosure does not specifically limit a connection method between the connection rib and the upper housing 11. Preferably, the connection rib and the upper housing 11 are fixedly connected by gluing, to enhance connection stability between the base 12 and the upper housing 11 and reduce a connection cost between the base 12 and the upper housing 11. In other embodiments, the connection rib may further be fixedly connected to the upper housing 11 by means of fasteners.

In other embodiments, the aperture 115 may further be formed by cutting the base 12, or by making an avoidance during molding of the base 12.

The present disclosure does not specifically limit a formation method of the sealing surface. Preferably, as illustrated in FIG. 1, FIG. 2 and FIG. 6, the bottom of the upper housing 11 has an edge 116 extending outward transversely and forming a sealing surface to increase area of the sealing surface, in such a manner that connection area between the seal 112 and the sealing surface is further increased. In this way, the sealing effect of the seal 112 on the first accommodation cavity 111 is further enhanced, and the difficulty of mounting the seal 112 is further reduced. In other embodiments, the sealing surface may further be formed from a bottom end surface of the upper housing 11.

Further, as illustrated in FIG. 1, FIG. 2 and FIG. 6, the upper housing 11 has a flange 117 at an outside of the upper housing 11. The base 12 is connected to the flange 117.

In an exemplary embodiment of the present disclosure, the flange 117 is located outside of the edge 116 to avoid interference between the edge 116 and the connection rib of the base 12. In addition, an arrangement of the flange 117 increases the connection area between the base 12 and the upper housing 11, which enhances connection stability between the base 12 and the upper housing 11, in such a manner that the product quality of the inserting apparatus is improved, and the use experience of the user is further improved. Besides, structural strength of the inserting apparatus is increased to ensure smoothness of the sensor of the sensor assembly 21 piercing into the subcutaneous tissue.

In other embodiments, the housing 1 may further be an integrally molded structure to increase structural strength of the housing 1.

In a preferred embodiment, as illustrated in FIG. 2 and FIG. 6, the housing 1 includes the upper housing 11 and the base 12 connected to the upper housing 11. The first accommodation cavity 111 is formed inside the upper housing 11. The second accommodation cavity 121 is formed inside the base 12. A mounting seat 122 configured for mounting of the transmitter assembly 22 is disposed inside the second accommodation cavity 121.

Since the transmitter assembly 22 is mounted at the mounting base 122, the stability of the transmitter assembly 22 is enhanced, and assembly of the transmitter assembly 22 is facilitated.

The present disclosure does not specifically limit a connection method between the transmitter assembly 22 and the mounting seat 122. Preferably, the transmitter assembly 22 has a periphery surface abutting against an inner wall of the mounting seat 122, that is, the transmitter assembly 22 is connected to the mounting seat 122 in the interference fit manner, to facilitate the transmitter assembly 22 to be separated from the mounting seat 122. In other embodiments, the transmitter assembly 22 may further be connected to the mounting seat 122 through an engagement structure.

Preferably, as illustrated in FIG. 2, FIG. 6 and FIG. 7, the mounting seat 122 has a limit engagement portion 123 located at a top of the transmitter assembly 22 and coming into contact with a top surface of the transmitter assembly 22, in such a manner that the limit engagement portion 123 can limit the transmitter assembly 22, that is, the transmitter assembly 22 is mounted at the mounting seat 122 from the bottom upward, to facilitate assembly of the sensor assembly 21 and facilitate the transmitter assembly 22 to be separated the mounting seat 122.

The present disclosure does not specifically limit the limit engagement portion 123. Preferably, as illustrated in FIG. 2, FIG. 6, and FIG. 7, the limit engagement portion 123 is an annular structure disposed in a circumferential direction of the transmitter assembly 22 to increase limit area for the transmitter assembly 22 and further enhance a limit effect on the transmitter assembly 22. In other embodiments, the limit engagement portion 123 may further be block structures arranged at intervals in the circumferential direction of the transmitter assembly 22.

Further, a top of the limit engagement portion 123 is spaced apart from the opening of the first accommodation cavity 111, in such a manner that an avoidance gap is formed between the top of the limit engagement portion 123 and the opening of the first accommodation cavity 111 to ensure the smooth removal of the seal 112.

Further, the base 12 has a contact surface at a bottom of the base 12, and the mounting seat 122 extends upward from the contact surface, in such a manner that after the transmitter assembly 22 is assembled to the mounting seat 122, the transmitter assembly 22 has a bottom end surface in flush with the contact surface. In addition, when the inserting apparatus is assembled to a user's body, the contact surface and the bottom end surface of the transmitter assembly 22 are both come in contact with skin of the user, to increase contact area between the inserting apparatus and the user and reduce discomfort caused to the user. In this way, the use experience of the user is improved.

Further, as illustrated in FIG. 1, FIG. 2, and FIG. 6, the housing 1 further includes a lower housing 13 connected to the base 12 and removable from the base 12 to expose the transmitter assembly 22.

In other words, the lower housing 13 can protect the transmitter assembly 22 from being damaged by the vibration during the transportation or the handling, to improve the quality stability of the inserting apparatus.

The present disclosure does not specifically limit a connection method between the lower housing 13 and the base 12. Preferably, the lower housing 13 is connected to the base 12 through an engagement structure, in such a manner that removal of the lower housing 13 is facilitated to improve the use experience of the user. In other embodiments, the lower housing 13 may further be connected to the base 12 by means of a threaded connection.

In a preferred embodiment, a height H of the aperture 115 and a thickness D of the seal 112 satisfy: H≥2D, which enables the seal to be removed through the aperture 115 more smoothly. In this way, the difficulty of removing the seal 112 is reduced, and the use experience of the user is further improved.

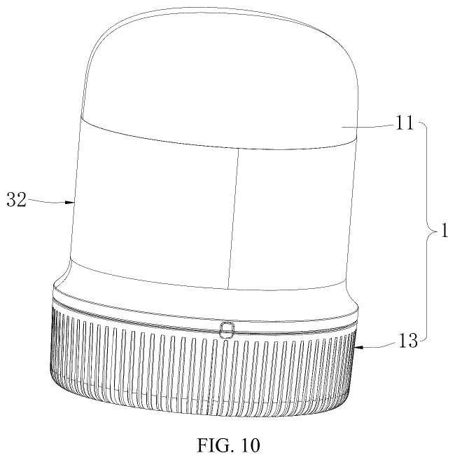

As illustrated in FIG. 10 to FIG. 20, the inserting apparatus for implantable sensor placement is provided. The inserting apparatus includes the housing 1, the monitoring unit 2, and the puncture assembly 5. The housing 1 includes the upper housing 11 and the lower housing 13 detachably connected to the upper housing 11. The monitoring unit 2 includes the transmitter assembly 22 disposed in the housing 1 and the sensor assembly 21 disposed at the transmitter assembly 22. The transmitter assembly 22 has an outer circumferential surface sealingly connected to an inner circumferential surface of the lower housing 13. A sealed cavity 138 is formed by the lower housing 13 and the transmitter assembly 22. The puncture assembly 5 includes the seat 51 disposed at the upper housing 11 and the needle 52 disposed at the seat 51. The needle 52 has an insertion portion arranged around the sensor assembly 21. Both the insertion portion and the sensor assembly 21 are located in the sealed cavity 138.

It should be understood that a part of the sensor assembly 21 needs to be pierced into the subcutaneous tissue of the human body to obtain monitoring data configured to characterize analyte concentration. In addition, the sensor assembly 21 is electrically connected to the transmitter assembly 22 to enable the sensor assembly 21 to analyze the analyte concentration obtained by the sensor assembly 21 and transmit the analyte concentration to a terminal.

Since both the insertion portion and the sensor assembly 21 are located in the sealed cavity 138, the sterilized needle 52 and the sensor assembly 21 are prevented from carrying the pathogenic bacteria again, in such a manner that a risk of the user being infected by pathogenic bacteria is reduced.

In addition, since the outer circumferential surface of the transmitter assembly 22 is sealingly connected to the inner circumferential surface of the lower housing 13, the sealed cavity 138 is formed by the lower housing 13 and the transmitter assembly 22, that is, the lower housing 13 and the transmitter assembly 22 jointly form a sterile barrier, which increases volume of the sterile barrier, in such a manner that accuracy of detecting tightness of the sterile barrier using a vacuum decay method can be increased. In this way, sealing detection methods of the inserting apparatus are increased, and the accuracy, reliability and convenience of detecting the tightness of the sterile barrier are increased.

In addition, since the outer circumferential surface of the transmitter assembly 22 is sealingly connected to the inner circumferential surface of the lower housing 13 to form the sealed cavity 138, a sealing position of the transmitter assembly 22 is set apart from an adhesive tape attached to a bottom of the transmitter assembly 22 to retain more complete adhesive tape area. In this way, a situation that the adhesive tape area is affected by an arrangement of the sterile barrier is avoided, ensuring stability of the monitoring unit 2 being adhered to the skin of the user.

The present disclosure does not specifically limit a sealing connection method between the outer circumferential surface of the transmitter assembly 22 and the inner circumferential surface of the lower housing 13, and any one of the following embodiments may be adopted.

Embodiment 1: in this embodiment, the lower housing 13 is integrally provided with a sealing rib 131 located at the inner circumferential surface of the lower housing 13 and extending in the circumferential direction of transmitter assembly 2. The sealing rib 131 has an inner circumferential surface sealingly connected to the outer circumferential surface of the transmitter assembly 22.

It should be understood that, in this embodiment, the sealing rib 131 is made of silicone gel, rubber, or other materials with sealing properties, in such a manner that the inner circumferential surface of the lower housing 13 is sealingly connected to the outer circumferential surface of the transmitter assembly 22 through the sealing rib 131.

Since the inner circumferential surface of the sealing rib 131 is sealingly connected to the outer circumferential surface of the transmitter assembly 22, volume of the sealed cavity 138 is further increased as compared to the inner circumferential surface of the lower housing 13 being directly sealingly connected to the outer circumferential surface of the transmitter assembly 22. In other words, the volume of the sterile barrier is further increased, which enables an effect of using the vacuum decay method to detect tightness of the inserting apparatus to be more obvious. In addition, while ensuring the seal between the outer circumferential surface of the transmitter assembly 22 and the inner circumferential surface of the lower housing 13, contact area between the inner circumferential surface of the lower housing 13 and an outer circumferential surface of a data output assembly is reduced compared to the outer circumferential surface of the transmitter assembly 22 being directly sealingly connected to the inner circumferential surface of the lower housing 13. In this way, a friction force between the transmitter assembly 22 and the lower housing 13 is reduced to ensure that the transmitter assembly 22 can be separated from the lower housing 13 when the lower housing 13 is removed, improving the use experience of the user.

It should be note that, the phrase “the lower housing 13 is integrally provided with the sealing rib 131 located at the inner circumferential surface of the lower housing 13” specifically means that the sealing rib 131 is non-detachably and fixedly connected to the inner circumferential surface of the lower housing 13. For example, the lower housing 13 and the sealing rib 131 are made by a two-color injection molding process to ensure the seal between the lower housing 13 and the sealing rib 131 and connection stability between the lowering housing 13 and sealing rib 131.

Embodiment 2: in this embodiment, a first seal 132 configured to sealingly connect the inner circumferential surface of the lower housing 13 to the outer circumferential surface of the transmitter assembly 22 is disposed between the inner circumferential surface of the lower housing 13 and the outer circumferential surface of the transmitter assembly 22, that is, the inner circumferential surface of the lower housing 13 and the outer circumferential surface of the transmitter assembly 22 are sealingly connected through the first seal 132. In this way, the seal between the transmitter assembly 22 and the lower housing 13 is enhanced, and tightness of the sealed cavity 138 is further ensured.

In this embodiment, a mounting method of the first seal 132 is not specifically limited, and any of the following examples may be adopted.

First example: in this example, the first seal 132 is fixedly connected to the inner circumferential surface of the lower housing 13, in such a manner that mounting of the transmitter assembly 22 is facilitated while ensuring the seal between the lower housing 13 and the transmitter assembly 22.

Second example: in this example, the first seal 132 is fixedly connected to the outer circumferential surface of the transmitter assembly 22, in such a manner that the mounting of the transmitter assembly 22 is facilitated while ensuring the seal between the transmitter assembly 22 and the lower housing 13.

Third example: in this example, the first seal 132 is only in close fit with the inner circumferential surface of the lower housing 13 and the outer circumferential surface of the transmitter assembly 22, that is, the inner circumferential surface of the lower housing 13 and the outer circumferential surface of the transmitter assembly 22 jointly squeeze the first seal 132 to fix the first seal 132.

Fourth example: in this example, one of the inner circumferential surface of the lower housing 13 and the outer circumferential surface of the transmitter assembly 22 has an annular groove along its circumferential direction. The first seal 132 is located in the annular groove and has a portion protruding from the annular groove. The portion of the first seal 132 protruding from the annular groove is in close contact with another of the inner circumferential surface of the lower housing 13 and the outer circumferential surface of the transmitter assembly 22, to facilitate the mounting of the transmitter assembly 22.

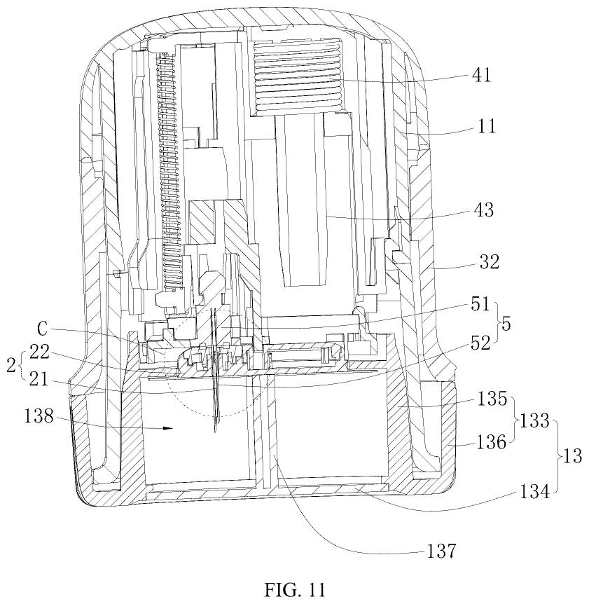

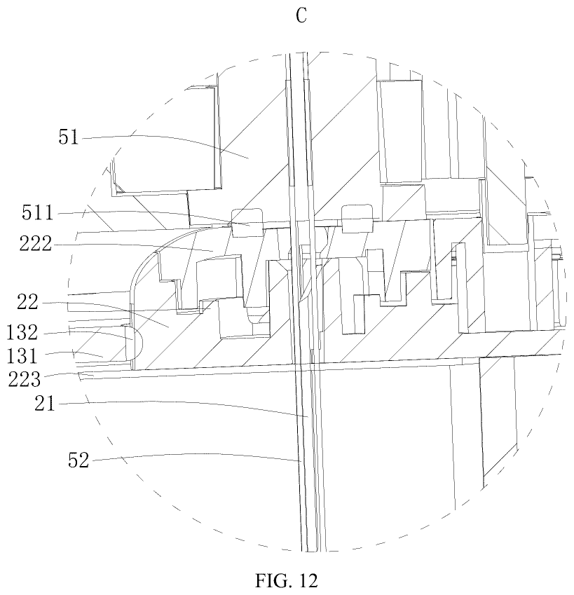

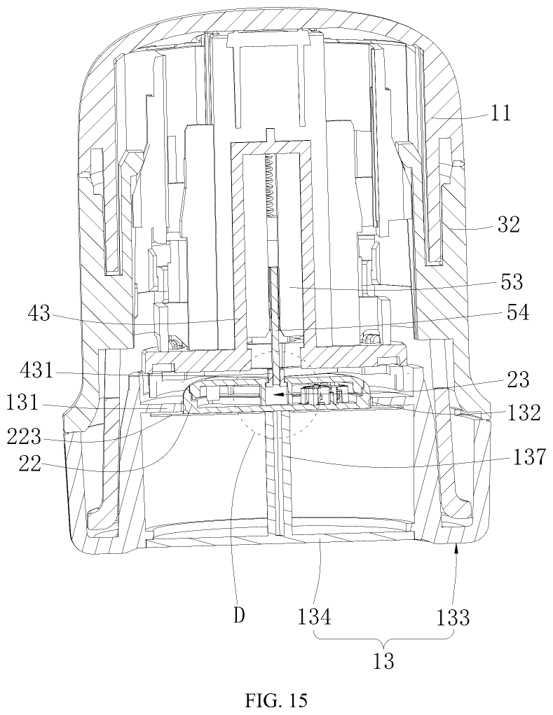

Embodiment 3: in this embodiment, as illustrated in FIG. 11, FIG. 12 and FIG. 15, the lower housing 13 is provided with the sealing rib 131 located at the inner circumferential surface of the lower housing 13 and extending in the circumferential direction of the transmitter assembly 22. The first seal 132 is disposed between the inner circumferential surface of the sealing rib 131 and the outer circumferential surface of the transmitter assembly 22 to seal the sealing rib 131 and the transmitter assembly 22.

It should be understood that the sealing rib 131 and the lower housing 13 are integrally molded to reduce a difficulty of molding the lower housing 13, in such a manner that a production cost of the lower housing 13 is reduced and a yield rate of the lower housing 13 is improved. The inner circumferential surface of the lower housing 13 is sealingly connected to the outer circumferential surface of the transmitter assembly 22 through the sealing rib 131 and the first seal 132, in such a manner that the volume of the sealed cavity 138 is further increased. In this way, the effect of detecting the tightness of the inserting apparatus by using the vacuum decay method is more obvious.

Preferably, the first seal 132 is fixedly connected to the sealing rib 131 to facilitate the mounting of the transmitter assembly 22 while ensuring the seal between the first seal 132 and the seal.

The present disclosure does not specifically limit a structure of the first seal 132. Preferably, the first seal 132 is an annular structure made of silicone gel, the rubber, and other materials with the sealing properties, to ensure the tightness of the sealed cavity 138. In other embodiments, the first seal 132 may further be a structure formed by solidification of a sealant disposed in the circumferential direction of the transmitter assembly 22.

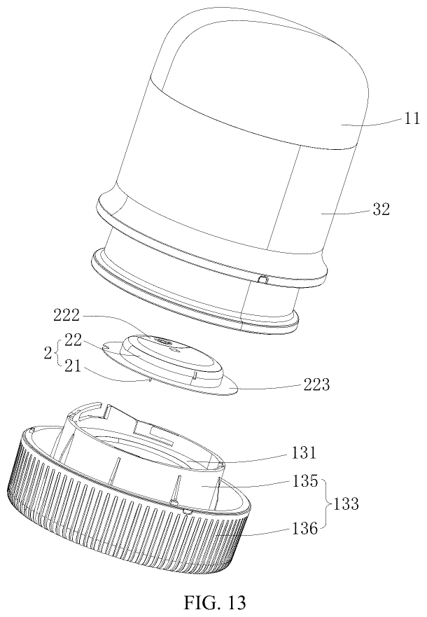

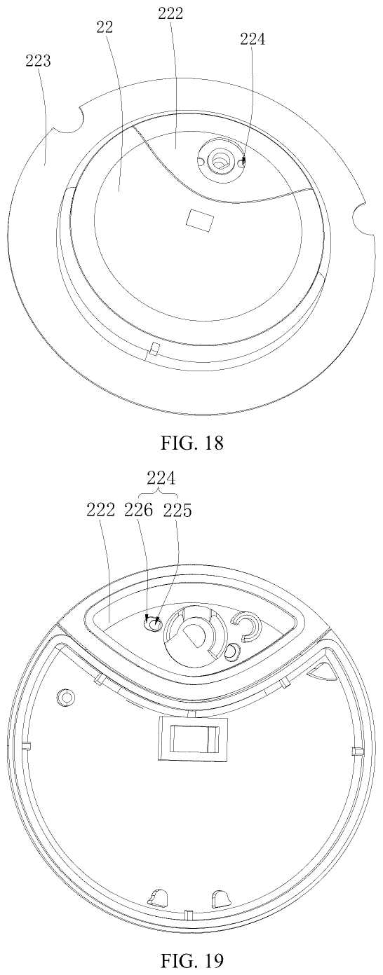

In a preferred embodiment, as illustrated in FIG. 12, FIG. 13, FIG. 17, FIG. 18, and FIG. 19, the transmitter assembly 22 has a cover body 222. A second seal 511 surrounding the needle 52 is disposed between the cover body 222 and the seat 51, in such a manner that the second seal 511 seals the cover body 222 and the seat 51, to further ensure the tightness of the sealed cavity 138, and further avoid contamination of the insertion portion and the sensor assembly 21 with the pathogenic bacteria.

The present disclosure does not specifically limit a structure of the second seal 511. Preferably, the second seal 511 is an annular structure made of the silicone gel, the rubber, and other materials with the sealing properties, to ensure the tightness of the sealed cavity 138. In other embodiments, the second seal 511 may further be a structure formed by the solidification of the sealant disposed in a circumferential direction of the seat 51.

The present disclosure does not specifically limit a mounting method of the second seal 511, and any one of the following examples may be adopted.

First example: in this example, as illustrated in FIG. 17, FIG. 18, and FIG. 19, the second seal 511 is provided with a limit connection portion 512. The cover body 222 has a limiting hole 224. The limit connection portion 512 has a connection segment 513 located in the limiting hole 224, and a stop segment 514 protruding from the limiting hole 224 and configured to abut against the cover body 222 upward.

In other words, after the second seal 511 is mounted at the cover body 222, the limit connection portion 512 is located in the limiting hole 224, and the stop segment 514 protrudes from the limiting hole 224 and abuts against the cover body 222 upward, in such a manner that the second seal 511 is limited through an engagement between the limit connection portion 512 and the limiting hole 224 to avoid the second seal 511 from being separated from the cover body 222. In this way, connection stability between the second seal 511 and the cover body 222 is enhanced. In addition, a situation that the second seal 511 falls off to cause panic to the user after the monitoring unit 2 is adhered to the skin of the user is avoided, ensuring the use experience of the user.

Further, as illustrated in FIG. 17, FIG. 18, and FIG. 19, the limiting hole 224 includes a mounting groove 225 and an accommodation groove 226. The mounting groove 225 is in communication with the accommodation groove 226, and the mounting groove 225 and the accommodation groove 226 are at least partially offset from each other. The stop segment 514 is configured to extend and protrude from the connection segment 513 in a radial direction. The connection segment 513 is located in the mounting groove 225, and the stop segment 514 is located in the accommodation groove 226.

It should be understood that the mounting groove 225 is located at a top of the accommodation groove 226. An opening of the mounting groove 225 and an opening of the accommodation groove 226 are at least partially offset from each other, in such a manner that a Z-shaped channel is formed by the mounting groove 225 and the accommodation groove 226. The connection segment 513 extends in a radial direction of the second seal 511, and the stop segment 514 is located at a bottom of the connection segment 513 and configured to extend and protrudes from the connection segment 513 in the radial direction, in such a manner that the connection segment 513 and the stop segment 514 are adapted to the mounting groove 225 and the accommodation groove 226, respectively, and then a Z-shaped step seal is formed between the limit connection portion 512 and the limiting hole 224, enabling a sealing path between the limit connection portion 512 and the limiting hole 224 to be more tortuous. In addition, sealing connection area between the limit connection portion 512 and the limiting hole 224 is increased, enhancing a sealing effect between the limit connection portion 512 and the limiting hole 224.

The present disclosure does not specifically limit the number of the limit connection portion 512 and the limiting hole 224. Preferably, as illustrated in FIG. 17, FIG. 18, and FIG. 19, a plurality of limit connection portions 512 and a plurality of limiting holes 224 are correspondingly provided in the circumferential direction of the seat 51, to further enhance the stability of the second seal 511. In other embodiments, both one stop connection portion 512 and one limiting hole 224 may further be provided.

Second example: in this example, the second seal 511 is fixedly connected to the seat 51, to avoid the situation that the second seal 511 falls off to cause panic to the user after the monitoring unit 2 is adhered to the skin of the user, ensuring the use experience of the user.

Third example: in this example, the second seal 511 is only in close fit with the seat 51 and the cover body 222, that is, the second seal 511 is fixed by mutual pressing of the seat 51 and the cover body 222, to reduce the production cost of the inserting apparatus.

In a preferred embodiment, the cover body 222 has an engagement groove surrounding the needle 52, and the second seal 511 is located in the engagement groove to reduce a thickness of the second seal 511 protruding from the cover body 222. In this way, a distance between the cover body 222 and the seat 51 is reduced to facilitate a miniaturization design of the inserting apparatus. In addition, the seal between the second seal 511 and the cover body 222 can be enhanced to further enhance the tightness of the sealed cavity 138.

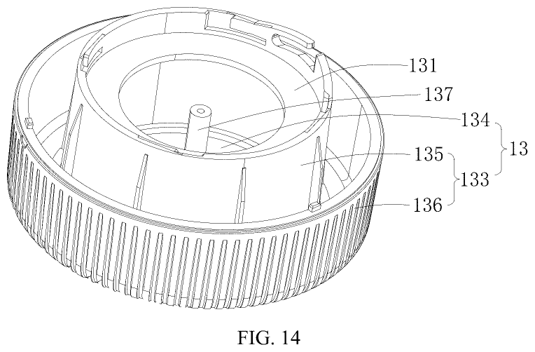

The present disclosure does not specifically limit a structure of the lower housing 13. Preferably, as illustrated in FIG. 11, FIG. 14, and FIG. 15, the lower housing 13 includes a body portion 133 and a sealing portion 134 detachably connected to the body portion 133. The sealing portion 134 is sealingly connected to the body portion 133 and has a support column 137 configured to abut with the transmitter assembly 22 and spaced apart from the needle 52. In this way, the support column 137 can support the transmitter assembly 22, avoiding a situation that the sealing disengagement is caused by the separation of the transmitter assembly 22 from the lower housing 13 due to the vibration of the inserting apparatus. Therefore, connection stability between the transmitter assembly 22 and the lower housing 13 is ensured, and the seal between the lower housing 13 and the transmitter assembly 22 is ensured. Since the support column 137 is spaced apart from the needle 52, the support column 137 avoids the needle 52 to avoid interference between the needle 52 and the support column 137. In this way, assembly of the inserting apparatus is facilitated to reduce the production cost of the inserting apparatus. In addition, since the lower housing 13 includes the body portion 133 and the sealing portion 134 that are provided separately, a difficulty of mounting the transmitter assembly 22 is thus reduced to improve the production efficiency of the inserting apparatus.

Preferably, as illustrated in FIG. 11 and FIG. 15, the support column 137 and the sealing portion 134 are coaxially arranged, and a central axis of the needle 52 and a central axis of the housing 1 are non-collinear arranged, ensuring an avoidance effect between the support column 137 and the needle 52.

It should be noted that, in this embodiment, the transmitter assembly 22 is disposed inside the body portion 133.

The present disclosure does not specifically limit a sealing connection method between the body portion 133 and the sealing portion 134. Preferably, the body portion 133 is sealingly connected to the sealing portion 134 by the sealant, to ensure the seal between the body portion 133 and the sealing portion 134 and reduce the production cost of the inserting apparatus. In other embodiments, a sealing ring is disposed between the body portion 133 and the sealing portion 134 to seal the body portion 133 and the sealing portion 134.

In other embodiments, the lower housing 13 may further be an integral structure.

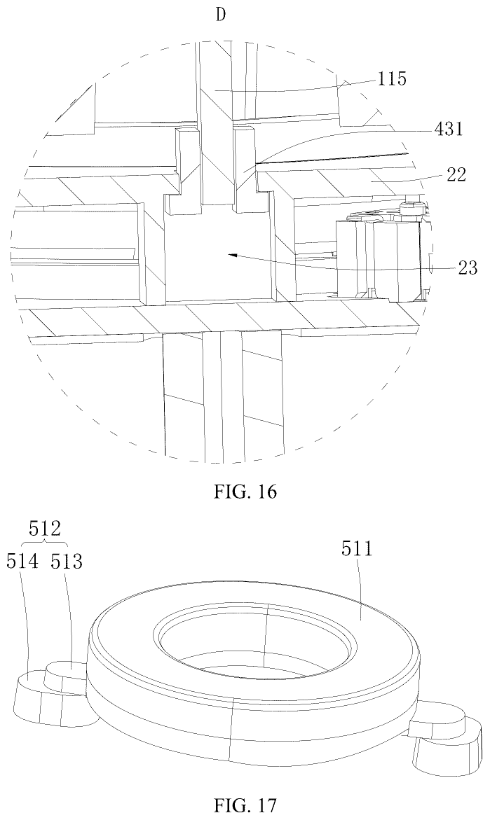

In a preferred embodiment, as illustrated in FIG. 15, FIG. 16, and FIG. 19, the transmitter assembly 22 has the limiting portion 23. The upper housing 11 has the core cylinder 43 and a sleeve 53. The core cylinder 43 has the locking member 431 extending into the limiting portion 23. The sleeve 53 has the fixing portion 54 acting on the locking member 431, to enable the locking member 431 to have the locked state in which the locking member 431 abuts against the groove wall of the limiting portion 23 and the unlocked state in which the locking member 431 is separated from the groove wall of the limiting portion 23.

It should be understood that, the seat 51 is disposed at the sleeve 53 to move with the sleeve 53, and the locking member 431 is elastic, in such a manner that the locking member 431 can be deformed under a pressing action of the fixing portion 54 and abuts against the groove wall of the limiting portion 23. In addition, after the fixing portion 54 is separated from the locking member 431, the locking member 431 can recover from the deformation and be separated from the groove wall of the limiting portion 23, realizing fixation and release of the transmitter assembly 22.

In other words, after the transmitter assembly 22 is mounted at the lower housing 13, the locking member 431 of the core cylinder 43 extends into the limiting portion 23, and the fixing portion 54 of the sleeve 53 presses the locking member 431, in such a manner that the locking member 431 is deformed and abuts against the groove wall of the limiting portion 23. At this time, the locking member 431 is in the locked state, allowing the transmitter assembly 22 to be connected to the core cylinder 43, in such a manner that the transmitter assembly 22 can follow the core cylinder 43 to move downward to perform an insertion action. After the insertion action is completed, the sleeve 53 moves upward, which enables the fixing portion 54 to be separated from the locking member 431, in such a manner that the locking member 431 recovers from the deformation under an elastic force action of the locking member 431. In this way, the locking member 431 is separated from the groove wall of the limiting portion 23. At this time, the locking member 431 is in the unlocked state, allowing the transmitter assembly 22 to be separated from the core cylinder 43, and then completing the process of adhering the monitoring unit 2 to the skin of the user. By connecting the transmitter assembly 22 to the core cylinder 43, the stability of the transmitter assembly 22 is enhanced to ensure the connection stability between the transmitter assembly 22 and the lower housing 13, in such a manner that the seal between the transmitter assembly 22 and the lower housing 13 is enhanced, and the tightness of the sealed cavity 138 is further ensured.

Further, as illustrated in FIG. 10, FIG. 11, FIG. 13, and FIG. 15, the core cylinder 43 is in a stop engagement with the upper housing 11. The upper housing 11 is provided with a trigger ring 32 arranged around the upper housing 11 and acting on the core cylinder 43. The upper housing 11 has the first drive member 41 acting on the core cylinder 43. When the trigger ring 32 moves downward relative to the upper housing 11, the core cylinder 43 releases the stop engagement with the upper housing 11 and moves downward under an action of the first drive member 41 to realize the insertion action.

Further, as illustrated in FIG. 11, FIG. 13, and FIG. 14, the lower housing 13 includes the body portion 133 and the sealing portion 134 sealingly connected to the body portion 133. The body portion 133 includes a sleeve 135 detachably connected to the upper housing 11 and a stop cylinder 136 arranged around the sleeve 135. The stop cylinder 136 is in a stop engagement with the trigger ring 32 in an axial direction of the upper housing 11 to avoid a situation that the trigger ring 32 triggers the core cylinder 43 when the lower housing 13 is not removed. In this way, the stability of the inserting apparatus during storage is ensured to improve the product quality of the inserting apparatus.

In a preferred embodiment, the lower housing 13 is provided with the sealing rib 131 located at the inner circumferential surface of the lower housing 13. The transmitter assembly 22 is provided with a fixing sticker 223 at the bottom of the transmitter assembly 22. The fixing sticker 223 extends to a bottom of the sealing rib 131 and comes into contact with the sealing rib 131.

Since the fixing sticker 223 extends to the bottom of the sealing rib 131, area of the fixing sticker 223 is increased to increase connection area between the fixing sticker 223 and the skin of the user, in such a manner that the stability of the monitoring unit 2 being adhered to the skin of the user is enhanced. In addition, since the fixing stick 223 comes into contact with the sealing rib 131, the seal between the transmitter assembly 22 and the lower housing 13 is further enhanced to greatly improve the tightness of the sealed cavity 138.

In other embodiments, as illustrated in FIG. 11 and FIG. 15, the fixing sticker 223 may further be spaced apart from the sealing rib 131.

When the inserting apparatus in the present disclosure is used, the lower housing 13 is first removed. At this time, the monitoring unit 2 is stably connected to the core cylinder 43 under an action of the locking member 431, and then the trigger ring 32 is slid downward to release the stop engagement between the core cylinder 43 and the upper housing 11. The core cylinder 43 moves downward under the action of the first drive member 41, in such a manner that the core cylinder 43 drives the sleeve 53 and the monitoring unit 2 to move downward to complete the insertion action, and then the sleeve 53 drives the needle 52 and fixing portion 54 to move upward. In this way, the fixing portion 54 is separated from the locking member 431, and the locking member 431 is released from abutting against and being engaged with the limiting portion 23, to complete a needle withdrawal action and realize separation of the monitoring unit 2 from the core cylinder 43.

The present disclosure does not specifically limit a structure of the fixing sticker 223. The fixing sticker 223 may be an adhesive tape configured to be adhered to the skin of the user or a sheet structure configured to be adhered to the skin of the user. As long as the monitoring unit 2 can be adhered to the skin of the user, the fixing sticker 223 can be any structure.

The present disclosure further discloses an assembling method of the inserting apparatus for implantable sensor placement, which is configured to assemble the inserting apparatus. The lowering housing 13 includes the body portion and the sealing portion 134 detachably connected to the body portion 133. The assembling method includes the following steps:

The body portion 133 is connected to the upper housing 11, that is, the body portion 133 is fixedly connected to the upper housing 11.

The transmitter assembly 22 is mounted at the body portion 133. At the same time, the transmitter assembly 22 is sealingly connected to the body portion 133, and the locking member 431 abuts against the groove wall of the limiting portion 23, in such a manner that mounting of the monitoring unit 2 is completed.

The sealing portion 134 is mounted at the body portion 133, in such a manner that the support column 137 of the sealing portion 134 abuts against the bottom of the transmitter assembly 22 to support the transmitter assembly 22. Then the sealing portion 134 and the body portion 133 are sealed, and finally the sealed cavity 138 is formed by the transmitter assembly 22 and the lower housing 13.

When the inserting apparatus is assembled, the body portion 133 of the lower housing 13 is first mounted at the upper housing 11, then the transmitter assembly 22 is mounted inside the body portion 133 and sealingly connected to the body portion 133, and then the sealing portion 134 is mounted. In this way, the support column 137 of the sealing portion 134 abuts against the bottom of the transmitter assembly 22 to support the transmitter assembly 22. Then the sealing portion 134 and the body portion 133 are sealed to enable the sealed cavity 138 to be formed by the transmitter assembly 22 and the lower housing 13, in such a manner that mounting of the inserting apparatus is completed. In this process, the difficulty of mounting the transmitter assembly 22 is reduced, in such a manner that assembly efficiency of the inserting apparatus is improved to reduce the production cost of the inserting apparatus.

In the description of the present disclosure, it should be understood that the orientation or the position indicated by terms such as “center”, “longitudinal”, “lateral”, “length”, “width”, “thickness”, “over”, “below”, “front”, “rear”, “left”, “right”, “vertical”, “horizontal”, “top”, “bottom”, “inner”, “outer”, “clockwise”, “anti-clockwise”, “axial”, “radial”, and “circumferential” should be construed to refer to the orientation or the position as shown in the drawings, and is only for the convenience of describing the present disclosure and simplifying the description, rather than indicating or implying that the pointed device or element must have a specific orientation, or be constructed and operated in a specific orientation, and therefore cannot be understood as a limitation of the present disclosure.

In addition, the terms “first” and “second” are only used for descriptive purposes, and cannot be understood as indicating or implying relative importance or implicitly indicating the number of indicated technical features. Therefore, the features associated with “first” and “second” may explicitly or implicitly include at least one of the features. In the description of the present disclosure, “plurality” means at least two, unless otherwise specifically defined.

In the present disclosure, unless otherwise clearly specified and limited, terms such as “install,” “connect,” “connect to,” “fix,” and the like should be understood in a broad sense. For example, it may be a fixed connection or a detachable connection or connection as one piece; mechanical connection or electrical connection or communication; direct connection or indirect connection through an intermediate; internal communication of two components or the interaction relationship between two components. For those of ordinary skill in the art, the specific meaning of the above-mentioned terms in the present disclosure can be understood according to specific circumstances.

In the present disclosure, unless expressly stipulated and defined otherwise, the first feature “on” or “under” the second feature may mean that the first feature is in direct contact with the second feature, or the first and second features are in indirect contact through another feature between them. Moreover, the first feature “above” the second feature means that the first feature is directly above or obliquely above the second feature, or simply means that the level of the first feature is higher than that of the second feature. The first feature “below” the second feature means that the first feature is directly below or obliquely below the second feature, or simply means that the level of the first feature is smaller than that of the second feature.

Reference throughout this specification to “an embodiment”, “some embodiments”, “an example”, “a specific example”, or “some examples” means that a particular feature, structure, material, or characteristic described in connection with the embodiment or example is included in at least one embodiment or example of the present disclosure. The appearances of the above phrases in various places throughout this specification are not necessarily referring to the same embodiment or example. Further, the particular features, structures, materials, or characteristics may be combined in any suitable manner in one or more embodiments or examples. In addition, different embodiments or examples and features of different embodiments or examples described in the specification may be combined by those skilled in the art without mutual contradiction.

Although embodiments of the present disclosure have been shown and described above, it should be understood that above embodiments are merely exemplary, and cannot be construed to limit the present disclosure. For those skilled in the art, various changes, modifications, replacements, and variations can be made to the embodiments without departing from the scope of the present disclosure.

What is claimed is:

1. An inserting apparatus for implantable sensor placement, comprising:
a housing;
a monitoring unit arranged in the housing and having a limiting portion; and
an insertion unit comprising a driving assembly, the driving assembly being configured to drive the monitoring unit to move and comprising a locking member configured to be engaged with the limiting portion, and the locking member having a locked state in which the locking member abuts with the limiting portion and an unlocked state in which the locking member is separated from the limiting portion.

2. The inserting apparatus according to claim 1, wherein the insertion unit comprises a puncture assembly, the puncture assembly comprises a fixing portion configured to apply a force towards the limiting portion to the locking member, to maintain the locking member in the locked state.

3. The inserting apparatus according to claim 2, wherein the housing has a first accommodation cavity and a second accommodation cavity located below the first accommodation cavity; and
the monitoring unit comprises a sensor assembly disposed in the first accommodation cavity and a transmitter assembly disposed in the second accommodation cavity, the limiting portion being provided at the sensor assembly.

4. The inserting apparatus according to claim 3, wherein the puncture assembly and the sensor assembly have a first relative position in which the puncture assembly and the sensor assembly are engaged with each other and a second relative position in which the puncture assembly and the sensor assembly are separated from each other, wherein:
in the first relative position, the fixing portion abuts against the locking member to restrict the locking member to move relative to the limiting portion; and
in the second relative position, the fixing portion is disengaged from the locking member to release a limitation on the locking member.

5. The inserting apparatus according to claim 3, wherein the transmitter assembly has a first engagement structure; and
the sensor assembly has a second engagement structure configured to be engaged with the first engagement structure, to connect the sensor assembly to the transmitter assembly.

6. The inserting apparatus according to claim 3, wherein the driving assembly comprises:
a first drive member configured to drive the puncture assembly to move from an initial position to an insertion position; and
a second drive member configured to drive the puncture assembly to move from the insertion position to a retraction position, to enable the fixing portion to be disengaged from the locking member.

7. The inserting apparatus according to claim 3, wherein a seal is disposed between the first accommodation cavity and the second accommodation cavity to seal the first accommodation cavity, the seal being removable through an aperture provided at a circumferential side of the housing, to enable the first accommodation cavity and the second accommodation cavity to be in communication with each other.

8. The inserting apparatus according to claim 2, wherein the locking member comprises at least two locking portions; and
the fixing portion is configured to extend between the at least two locking portions, to prevent the at least two locking portions from moving relative to the limiting portion.

9. The inserting apparatus according to claim 1, wherein the locking member is an elastic member and is in a deformed state when the locking member is in the locked state.

10. The inserting apparatus according to claim 1, wherein the locking member comprises a connection segment and a locking segment disposed at a circumferential side of the connection segment;
the limiting portion comprises a connection groove configured to accommodate the connection segment, and an engagement groove radially recessed from a groove wall of the connection groove and configured to accommodate the locking segment;
in the locked state, the locking segment is located inside the engagement groove; and
in the unlocked state, the locking segment is disengaged from the engagement groove.

11. The inserting apparatus according to claim 1, wherein the limiting portion has a contact surface engaged with the locking member, the contact surface being inclined from a center to a periphery in a movement direction of the monitoring unit.

12. An inserting apparatus for implantable sensor placement, comprising:
a housing having a first accommodation cavity and a second accommodation cavity located below the first accommodation cavity, wherein the housing has an aperture provided at a circumferential side of the housing and in communication with the first accommodation cavity and/or the second accommodation cavity;
a monitoring unit comprising a sensor assembly disposed in the first accommodation cavity and a transmitter assembly disposed in the second accommodation cavity;
a driving assembly configured to drive the monitoring unit to move and comprising a locking member configured to prevent the monitoring unit from being disengaged from the driving assembly; and
a seal configured to seal the first accommodation cavity, the seal being removable through the aperture to unseal the first accommodation cavity.

13. The inserting apparatus according to claim 12, wherein the seal comprises a manipulating portion extending to an outside of the housing via the aperture.

14. The inserting apparatus according to claim 13, wherein the seal is a diaphragm structure bonded to an opening of the first accommodation cavity; and
the manipulating portion extends from the aperture to the outside of the housing.

15. The inserting apparatus according to claim 12, wherein the housing comprises an upper housing and a base connected to the upper housing, wherein:
the first accommodation cavity is formed inside the upper housing;
the second accommodation cavity is formed inside the base; and
the upper housing has a sealing surface at a bottom of the upper housing, the seal being connected to the sealing surface to seal the first accommodation cavity.

16. The inserting apparatus according to claim 15, wherein the base has a connection area and an avoidance area in a circumferential direction of the base, wherein:
the connection area is provided with a connection rib connected to the upper housing; and
the aperture is formed between the avoidance area and the upper housing.

17. The inserting apparatus according to claim 12, wherein a height H of the aperture and a thickness D of the seal satisfy: H≥2D.

18. An inserting apparatus for implantable sensor placement, comprising:
a housing comprising an upper housing and a lower housing detachably connected to the upper housing;
a monitoring unit having an outer circumferential surface sealingly connected to an inner circumferential surface of the lower housing, a sealed cavity being formed by the lower housing and the monitoring unit;
a puncture assembly comprising a seat and a needle disposed at the seat, the needle having an insertion portion configured around the sensor assembly, both the insertion portion and the sensor assembly being located in the sealed cavity; and
a driving assembly configured to drive the puncture assembly and the monitoring unit to move.

19. The inserting apparatus according to claim 18, wherein:
the lower housing is provided with a sealing rib located at the inner circumferential surface and extending in a circumferential direction of the monitoring unit; and
the sealing rib has an inner circumferential surface sealingly connected to the outer circumferential surface of the monitoring unit.

20. The inserting apparatus according to claim 18, wherein:
the transmitter assembly has a limiting portion;
the upper housing has a core cylinder and a sleeve;
the driving assembly is configured to drive the sleeve and the monitoring unit to move through the core cylinder;
the core cylinder has a locking member configured to extend into the limiting portion; and
the sleeve has a fixing portion acting on the locking member, to enable the locking member to have a locked state in which the locking member abuts against the limiting portion and an unlocked state in which the locking member is separated from the limiting portion.
